(19) United States
(12) Patent Application Publication
(10) Pub. No.: US 2010/0030302 A1
(43) Pub. Date: Feb. 4, 2010

(54) METHOD FOR DISPLAYING TRENDED DATA RETRIEVED FROM A MEDICAL DEVICE

(75) Inventors: Paul A. Blowers, Minneapolis, MN (US); Bohdan O. Washchuk, La Grange, IL (US); Scott W. Davie, Saint Paul, MN (US); Laurie K. Schumacher, Crystal, MN (US); Keith Florance, New York, NY (US); Joel R. Lauer, Rogers, MN (US); JoAnn Abraham, Coon Rapids, MN (US); James E. Willenbring, Saint Pal, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: MEDTRONIC, INC., Minneapolis, MN (US)

(21) Appl. No.: 12/182,620

(22) Filed: Jul. 30, 2008

(51) Int. Cl. A61N 1/08; G06Q 50/00

(52) U.S. Cl. 607/60; 705/2

(57) ABSTRACT

A system for displaying trended data retrieved from a medical device comprising a medical device which monitors and stores trended patient data, a data center, and an intermediate data relay device. The intermediate device receives stored trended patient data from the medical device and uploads the data to the data center. The data center merges the uploaded data with patient data obtained during previous data uploads. Clinicians can access the uploaded and merged patient using an interactive web page which allows the clinicians to seamlessly view the data.

### BACKGROUND

There are numerous devices which collect medical data about a patient over time. An example of such devices are IMDs (Implantable Medical Devices), which are implanted within a patient and measure and record data. The data stored in the IMD is then periodically downloaded from the device for evaluation by a clinician. Other medical devices which are not implanted may similarly collect clinical data, such as hand held glucose monitors.

The data supplied by these devices allow clinicians to monitor measurable patient variables, such as cardiac data. Using this data, the clinicians are able to make decisions about the course of treatment or the need for various interventions. While there is an ability to collect large amounts of patient data, such data is most valuable for clinical decision making when it is available to a clinician in a format which is useful, easy to interpret and monitor over time, and which can be easily manipulated to suit the needs of the clinician.

One system for monitoring data relating to cardiac performance is the CareLink® system provided by Medtronic, Inc. In this system, an IMD monitors and stores cardiac data. The data is stored in packets and the packets are periodically uploaded to a programmer, such as once every 2 weeks. The CareLink® system allows the data to be viewed by clinicians using a web based system. However, the data of each upload is not integrated with previously uploaded data. As a result, the data is only available to the clinician in a piecemeal fashion. For example, if the clinician is viewing one month of data, he or she cannot move the viewer directly ahead to the next month of data without navigating up to a one year view of the data and then back down to the desired month. As a result, while the system provides the clinician with access to the data, the lack of data integration can make it difficult for the clinician to use and interpret the data.

### BRIEF DESCRIPTION OF THE DRAWINGS

The following drawings are illustrative of particular embodiments of the present invention and therefore do not limit the scope of the invention. The drawings are intended for use in conjunction with the explanations in the following detailed description. Embodiments of the present invention will hereinafter be described in conjunction with the appended drawings, wherein like numerals denote like elements.

FIG. 1 is a schematic diagram depicting a multi-channel, atrial and bi-ventricular, monitoring/pacing implantable medical device (IMD) in which embodiments of the invention may be implemented;

FIG. 2 is a simplified block diagram of an embodiment of IMD circuitry and associated leads that may be employed in the system of FIG. 1 to enable selective therapy delivery and monitoring in one or more heart chamber;

FIG. 3 is a simplified block diagram of a single monitoring and pacing channel for acquiring pressure, impedance and cardiac EGM signals employed in monitoring cardiac function and/or delivering therapy, including pacing therapy, in accordance with embodiments of the invention;

FIG. 4 is a screen shot of a webpage displaying trended cardiac data in accordance with embodiments of the invention;

FIG. 5 is another screen shot of a webpage displaying trended cardiac data in accordance with embodiments of the invention;

FIG. 6 is another screen shot of a webpage displaying trended cardiac data in accordance with embodiments of the invention; and

FIG. 7 is another screen shot of a webpage displaying trended cardiac data in accordance with embodiments of the invention.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention.

Embodiments of the invention comprise a system and method for displaying trended data retrieved from a medical device. The trended data is displayed as a webpage which is accessible to clinicians from any computer in a form which is user friendly and can be easily manipulated by the clinician. Typically the trended data is obtained by an implantable medical device (IMD) though other medical device such as handheld devices like glucose monitors can also be used as a source of data for the data display.

Implantable medical devices (IMDs) which monitor and deliver therapy to a patient's heart may be used with the invention. IMDs typically sense a patient's cardiac electrogram, interpret the electrogram to represent a cardiac rhythm, and deliver therapy based on that interpretation. Accurate electrical sensing and data interpretation are therefore essential to the delivery of appropriate therapy by the IMDs. Embodiments of this invention employ intracardiac pressure data for monitoring cardiac activity. Such pressure data may be used alone for patient monitoring or in conjunction with EGM, such as to confirm the accurate interpretation of EGM data. Some embodiments may measure impedance values, such as for providing a measure of lung wetness as an indication of volume status or volume overload. Certain embodiments of the invention may include, or may be adapted for use in, diagnostic monitoring equipment, external medical device systems, and implantable medical devices (IMDs), including implantable hemodynamic monitors (IHMs), implantable cardioverter-defibrillators (ICDs), cardiac pacemakers, cardiac resynchronization therapy (CRT) pacing devices, leadless pacing devices, drug delivery devices, or combinations of such devices.

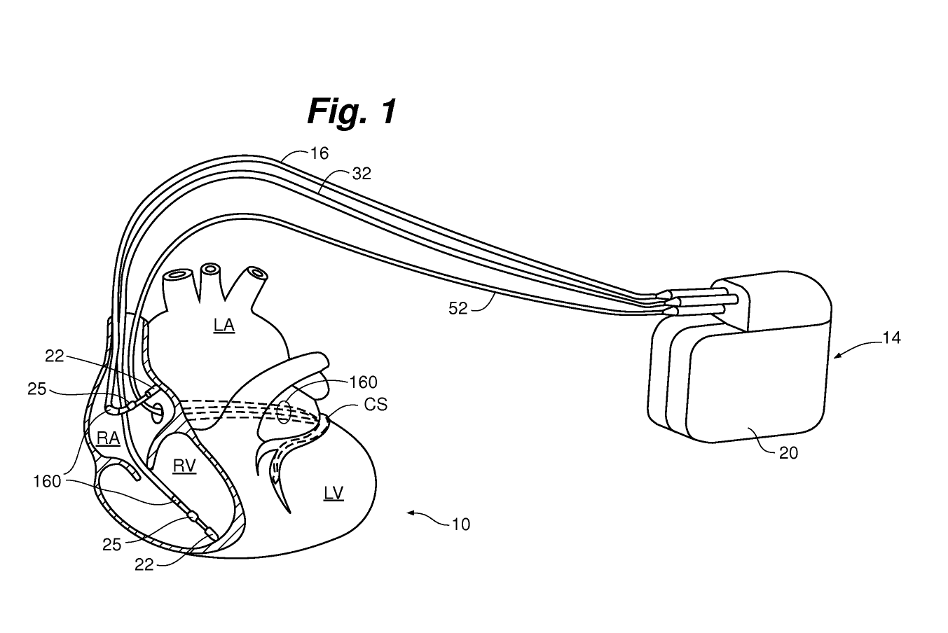

FIG. 1 is a schematic representation of an implantable medical device (IMD) 14 that may be used in accordance with certain embodiments of the invention. The IMD 14 may be any device that is capable of measuring hemodynamic parameters (e.g., blood pressure signals) from within a ventricle of a patient's heart, and which may further be capable of measuring other signals, such as the patient's electrogram (EGM).

In FIG. 1, heart 10 shows the right atrium (RA), left atrium (LA), right ventricle (RV), left ventricle (LV), and the coronary sinus (CS) extending from the opening in the right atrium laterally around the atria to form the great vein.

FIG. 1 depicts IMD 14 in relation to heart 10. In certain embodiments, IMD 14 may be an implantable, multi-channel cardiac pacemaker that may be used for restoring AV synchronous contractions of the atrial and ventricular chambers and simultaneous or sequential pacing of the right and left ventricles. Three endocardial leads 16, 32 and 52 connect the IMD 14 with the RA, the RV and the LV, respectively. Each lead has at least one electrical conductor and pace/sense electrode, and a can electrode 20 may be formed as part of the outer surface of the housing of the IMD 14. The pace/sense electrodes and can electrode 20 may be selectively employed to provide a number of unipolar and bipolar pace/sense electrode combinations for pacing and sensing functions. The depicted positions in or about the right and left heart chambers are merely exemplary. Moreover other leads and pace/sense electrodes may be used instead of the depicted leads and pace/sense electrodes.

It should be noted that the IMD 14 may also be an implantable cardioverter defibrillator (ICD), a cardiac resynchronization therapy (CRT) device, an insertable loop recorder (ILR), an implantable hemodynamic monitor (IHM), or any other such device or combination of devices, according to various embodiments of the invention.

Typically, in pacing systems of the type illustrated in FIG. 1, the electrodes designated above as “pace/sense” electrodes are used for both pacing and sensing functions. In accordance with one aspect of the present invention, these “pace/sense” electrodes can be selected to be used exclusively as pace or sense electrodes or to be used in common as pace/sense electrodes in programmed combinations for sensing cardiac signals and delivering pace pulses along pacing and sensing vectors.

In addition, some or all of the leads shown in FIG. 1 could carry one or more pressure sensors for measuring systolic and diastolic pressures, as well as incorporating electrodes which are spaced apart which function as impedance sensing leads for deriving volumetric measurements of the patient's torso and expansion and contraction of the RA, LA, RV and LV.

The leads and circuitry described above can be employed to record EGM signals, blood pressure signals, impedance values and other variables over certain time intervals. The recorded data may be periodically telemetered out to a programmer operated by a physician or other healthcare worker in an uplink telemetry transmission during a telemetry session, for example. Alternatively, the data may be telemetered to a personal data monitor, which may be done at the patient's home. This uplink to a personal data monitor may be performed at periodic intervals by the patient himself or herself (such as by holding an antenna over the device) or may occur automatically at specific times, such as at intervals programmed by a clinician. The uplink of data to the personal data monitor may also be initiated by the IMD upon detection of an event, such as atrial fibrillation, delivery of a shock, or a device integrity issue.

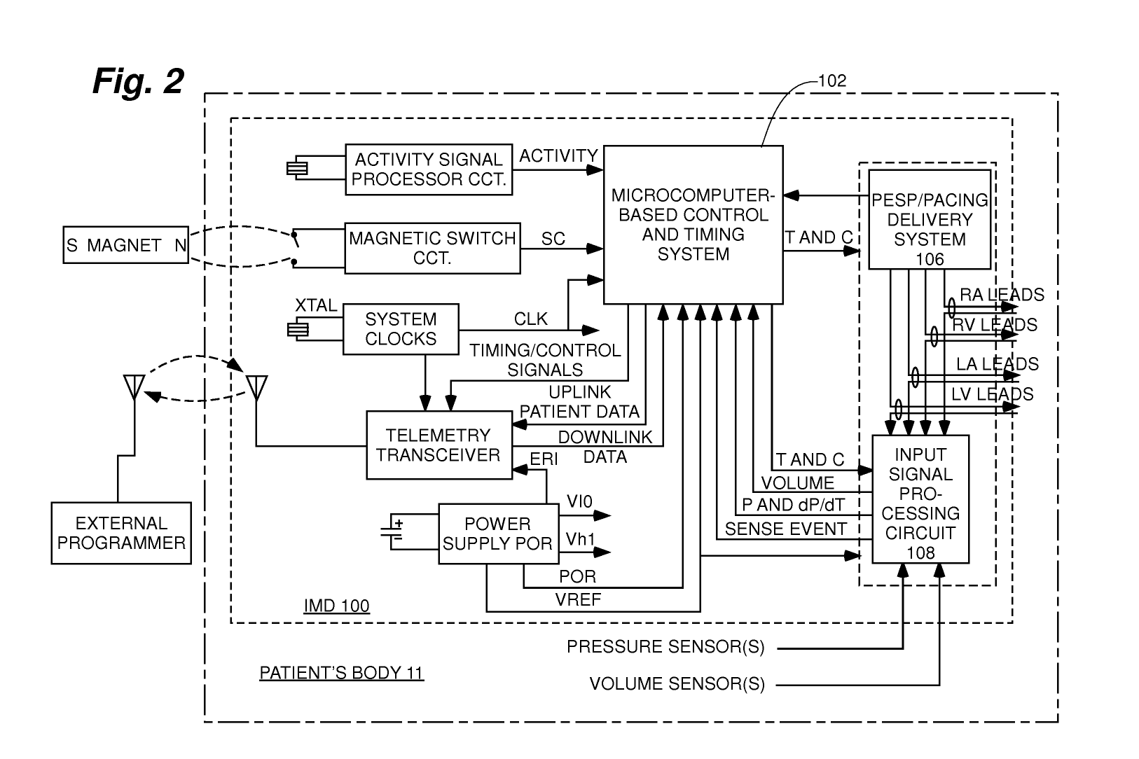

FIG. 2 depicts a system architecture of an exemplary multi-chamber monitor/sensor 100 implanted into a patient's body 11 that provides delivery of a therapy and/or physiologic input signal processing. The typical multi-chamber monitor/sensor 100 has a system architecture that is constructed about a microcomputer-based control and timing system 102 which varies in sophistication and complexity depending upon the type and functional features incorporated therein. The functions of microcomputer-based multi-chamber monitor/sensor control and timing system 102 are controlled by firmware and programmed software algorithms stored in RAM and ROM including PROM and EEPROM and are carried out using a CPU or ALU of a typical microprocessor core architecture.

The therapy delivery system 106 can be configured to include circuitry for delivering cardioversion/defibrillation shocks and/or cardiac pacing pulses delivered to the heart or cardiomyostimulation to a skeletal muscle wrapped about the heart. Alternately, the therapy delivery system 106 can be configured as a drug pump for delivering drugs into the heart to alleviate heart failure or to operate an implantable heart assist device or pump implanted in patients awaiting a heart transplant operation.

The input signal processing circuit 108 includes at least one physiologic sensor signal processing channel for sensing and processing a sensor derived signal from a physiologic sensor located in relation to a heart chamber or elsewhere in the body. Examples illustrated in FIG. 2 include pressure and volume sensors, but could include other physiologic or hemodynamic sensors.

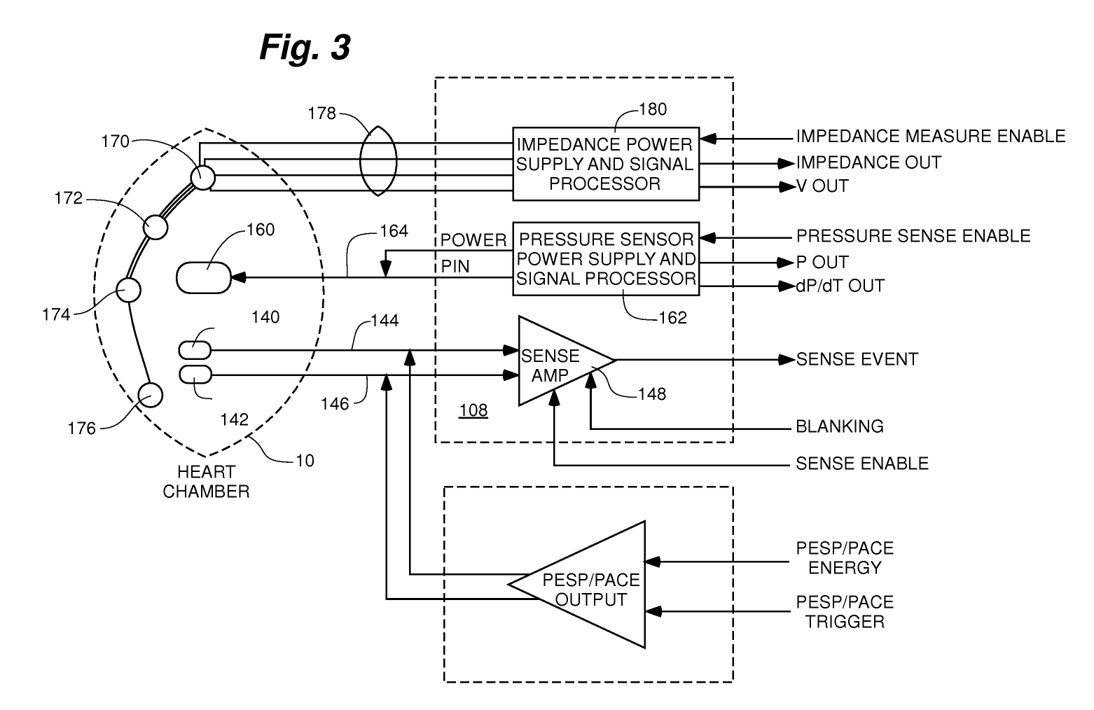

FIG. 3 schematically illustrates one pacing, sensing and parameter measuring channel in relation to one heart chamber. A pair of pace/sense electrodes 140, 142, a pressure sensor 160, and a plurality, e.g., four, impedance measuring electrodes 170, 172, 174, 176 are located in operative relation to the heart 10.

The pair of pace/sense electrodes 140, 142 are located in operative relation to the heart 10 and coupled through lead conductors 144 and 146, respectively, to the inputs of a sense amplifier 148 located within the input signal processing circuit 108. The sense amplifier 148 is selectively enabled by the presence of a sense enable signal that is provided by control and timing system 102. The sense amplifier 148 is enabled during prescribed times when pacing is either enabled or not enabled in a manner known in the pacing art. The blanking signal is provided by control and timing system 102 upon delivery of a pacing or PESP pulse or pulse train to disconnect the sense amplifier inputs from the lead conductors 144 and 146 for a short blanking period in a manner well known in the art. The sense amplifier provides a sense event signal signifying the contraction of the heart chamber commencing a heart cycle based upon characteristics of the EGM. The control and timing system responds to non-refractory sense events by restarting an escape interval (EI) timer timing out the EI for the heart chamber, in a manner well known in the pacing art.

The pressure sensor 160 is coupled to a pressure sensor power supply and signal processor 162 within the input signal processing circuit 108 through a set of lead conductors 164. Lead conductors 164 convey power to the pressure sensor 160, and convey sampled blood pressure signals from the pressure sensor 160 to the pressure sensor power supply and signal processor 162. The pressure sensor power supply and signal processor 162 samples the blood pressure impinging upon a transducer surface of the sensor 160 located within the heart chamber when enabled by a pressure sense enable signal from the control and timing system 102. Absolute pressure (P), developed pressure (DP) and pressure rate of change (dP/dt) sample values can be developed by the pressure sensor power supply and signal processor 162 or by the control and timing system 102 for storage and processing.

A variety of hemodynamic parameters may be recorded, for example, including right ventricular (RV) systolic and diastolic pressures (RVSP and RVDP), estimated pulmonary artery diastolic pressure (ePAD), pressure changes with respect to time (dP/dt), heart rate, activity, and temperature. Some parameters may be derived from others, rather than being directly measured. For example, the ePAD parameter may be derived from RV pressures at the moment of pulmonary valve opening, and heart rate may be derived from information in an intracardiac electrogram (EGM) recording.

The set of impedance electrodes 170, 172, 174 and 176 is coupled by a set of conductors 178 and is formed as a lead that is coupled to the impedance power supply and signal processor 180. Impedance-based measurements of cardiac parameters such as stroke volume are known in the art, such as an impedance lead having plural pairs of spaced surface electrodes located within the heart 10. The spaced apart electrodes can also be disposed along impedance leads lodged in cardiac vessels, e.g., the coronary sinus and great vein or attached to the epicardium around the heart chamber. The impedance lead may be combined with the pace/sense and/or pressure sensor bearing lead.

The data stored by IMD 14 may include continuous monitoring of various parameters, for example recording intracardiac EGM data at sampling rates as fast as 256 Hz or faster. In certain embodiments of the invention, an IHM may alternately store summary forms of data that may allow storage of data representing longer periods of time. In one embodiment, hemodynamic pressure parameters may be summarized by storing a number of representative values that describe the hemodynamic parameter over a given storage interval. The mean, median, an upper percentile, and a lower percentile are examples of representative values that may be stored by an IHM to summarize data over an interval of time (e.g., the storage interval). In one embodiment of the invention, a storage interval may contain six minutes of data in a data buffer, which may be summarized by storing a median value, a 94th percentile value (i.e., the upper percentile), and a 6th percentile value (i.e., the lower percentile) for each hemodynamic pressure parameter being monitored. In this manner, the memory of the IHM may be able to provide weekly or monthly (or longer) views of the data stored. The data buffer, for example, may acquire data sampled at a 256 Hz sampling rate over a 6 minute storage interval, and the data buffer may be cleared out after the median, upper percentile, and lower percentile values during that 6 minute period are stored. It should be noted that certain parameters measured by the IHM may be summarized by storing fewer values, for example storing only a mean or median value of such parameters as heart rate, activity level, and temperature, according to certain embodiments of the invention.

Hemodynamic parameters that may be used in accordance with various embodiments of the invention include parameters that are directly measured, such as RVDP and RVSP, as well as parameters that may be derived from other pressure parameters, such as estimated pulmonary artery diastolic pressure (ePAD), rate of pressure change (dP/dt), etc.

The IMDs described above provide hemodynamic monitoring and therapy to a patient's heart. Other types of IMDs which collect data over time are also useful in embodiments of the invention, such as glucose monitors, and neurological stimulators.

After the patient data is retrieved from the IMD by a relay communication device such as a programmer or a personal data monitor, it is sent to a remote internet web based data center. The data center represents a high speed computer network system which is located remotely and which stores and processes the data from the IMD. The programmer, personal data monitor, or other relay communication device therefore functions as an interface between the IMD and the data center. This communication between the programmer and personal data monitor and the data center may occur using standard internet connections such as telephone or cable lines or may use wireless data communications links or any other form of telecommunication.

Each time IMD data is uploaded to the data center, the data typically corresponds to a particular time period. The time period is typically the time since the most recent data upload. Data uploads may occur daily, weekly, or at other periodic or aperiodic intervals. The data center processes the uploaded data to merge the new data with data obtained during previous uploads. As a result, the data is not limited to separate uploads but rather is combined and integrated. The stored data is therefore continuous (continguous) over time, including all current and previously uploaded data.

After data is telemetered from an IMD to a relay communication device such as a programmer or personal data monitor and then sent to a data center, the data may be accessed by clinicians through the internet using any computer terminal. A website is provided by the data center for viewing patient IMD data. The clinician accesses the website by logging onto the site using a remote work station such as a personal computer and requests to see the data for a specific patient. Data is provided to the clinician using an interactive webpage. Because patient data is integrated by the data center, patient data may be seamlessly supplied to the website without requiring the user to navigate through different time windows or unique date specific links to reach a desired view.

Upon opening, the website first provides a default view of IMD data trends over time. In some embodiments, the default view is the most recent two weeks of data trends. In some embodiments, the clinician may customize the default view to any desired time period.

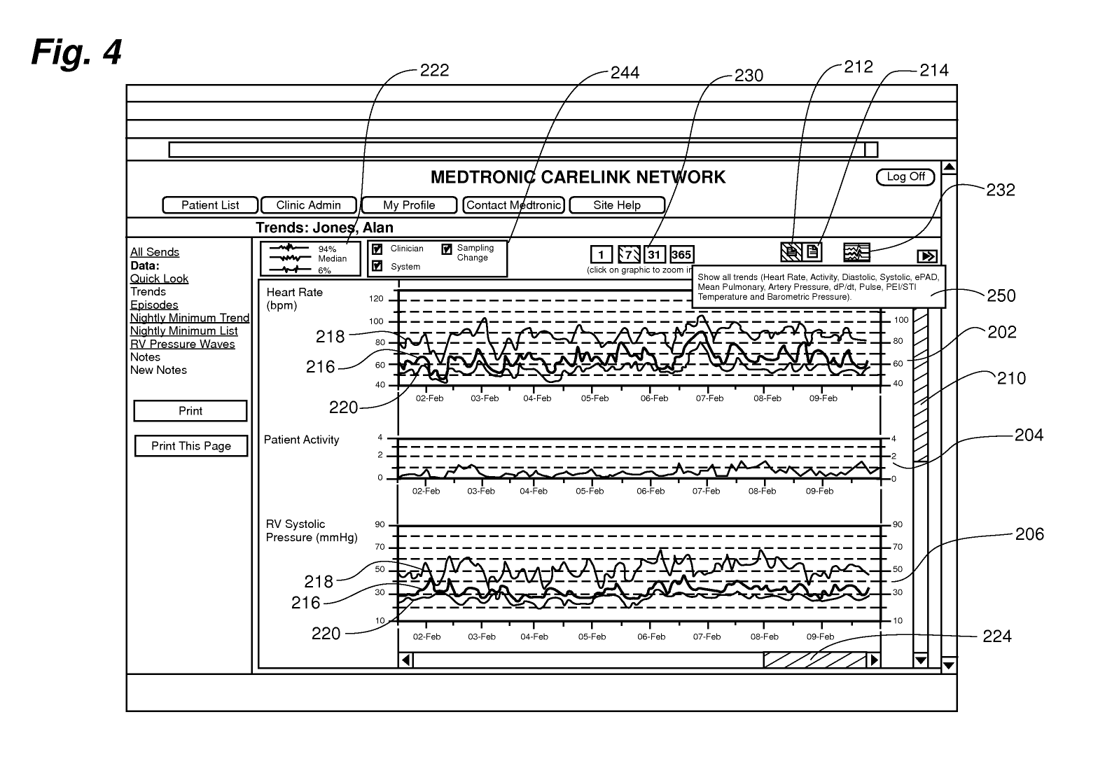

An example of a web page displaying a default view is shown in FIG. 4. The default view displays a set of primary trends. In this example, there are six primary trends including the three trends, heart rate 202, patient activity 204, and RV systolic pressure 206, shown in FIG. 4. The trends are displayed horizontally as a graph with correlating data for each trend shown over time, with numerical values on the y axis and time on the x-axis. When multiple trends are provided, as in FIG. 1, not all primary trends are visible on the page at one time; therefore a vertical scroll bar 210 allows the clinician to scroll up and down to view the other trends. Additional primary trends include, for example, (RV) diastolic pressures (RVDP), estimated pulmonary artery diastolic pressure (ePAD), pressure changes with respect to time (dP/dt), pulse, PEI/STI, pacing capture data, electrical device data, temperature, and Barometric pressure. The choice of which trends are provided in the default view may be customized for each patient and/or each clinician viewing the patient data.

The website can also optionally include secondary trends. Secondary trends may include trends for device troubleshooting, derived trends, and trends for research interest, for example. The clinician may select between viewing primary trends only or primary trends and secondary trends by clicking on webpage buttons representative of either view. As shown in FIG. 4, the primary trend button 212 looks like a short list and this button directs the website to display only primary trends. A primary and secondary trend button 214, adjacent to the primary trend button 212, looks similar to the primary trend button 212 but the symbol shows a longer list. This button directs what website to display both primary trends and secondary trends. The primary trend button 212 appears to be depressed in FIG. 4, indicating that only the primary trends are provided on the webpage. In some embodiments, the clinician or the clinic may select the primary and/or secondary trends to be viewed based on user preferences of the role of the clinician. For example, the clinician may select the trends based upon the patient's condition (such as heart failure) or based upon the clinician's role (such as cardiologist or electrophysiologist).

The data presented in the trend lines may be displayed in a variety of ways. For example, the measured values across time may be shown. Alternatively, when the data is in summary form, the mean value may be displayed. In addition, different percentile values could be shown such as the 6^{th} and 94^{th} percentile or standard deviations. Each of the mean value and the percentile values could be presented distinctly, such as by using different types of lines or different colors of lines. In the example shown in FIG. 4, the heart rate data 202 is provided with the mean 216 as the center and is the darkest line. The 94^{th} percentile line 218 is above the mean line 216 and the 6^{th} percentile line 220 is below the mean line 216. A trend line legend 222 is also provided on the webpage to identify the type of data shown in the trend line. Alternatively, the clinician may be able to select the type of trend line data displayed, such as by toggling between the types of trend line data by clicking a button on the screen.

The data presented in the trend lines is shown in graphical form. The clinician can estimate the value of a particular data point using the x-axis as a guide. Alternatively, the data may be presented with a roll-over feature. When the clinician hovers the cursor over a point on the data trend, a window appears revealing the numerical value of the data point, allowing the clinician to obtain a precise value.

In order to allow quicker scrolling across time, the website may download additional data along with the data visible on the screen. For example, the default view may be a 1 week view and may display the data from the most recent one week period. However, the website may also download data from the previous one week, so that two weeks of data are downloaded for the one week view. In this way, the clinician can scroll back from the current week to the previous week without having to wait for the additional data to download to facilitate data analysis. In the same way, a one month view may be downloaded with the data from the previous month. Thus, for each view, data is downloaded not only for the time period being viewed but also for an additional time period equal to the time period being viewed. In this way, the data is continuously available without delay over a time period which is greater than that which is shown on the computer screen.

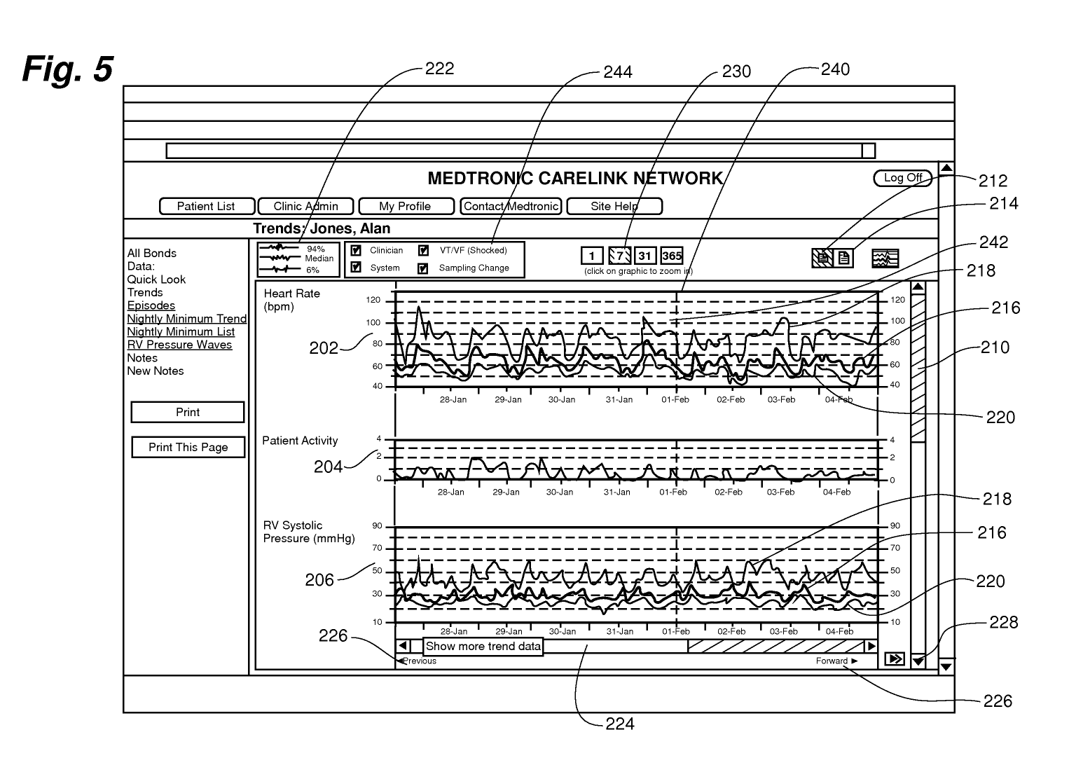

The clinician may scroll across time using a horizontal scroll bar 224. In the example shown in FIG. 4, the horizontal scroll bar 224 is at the bottom of the page, beneath those trend lines which are visible on the page. In order to scroll horizontally beyond the data available on the page, the clinician must append additional data. This may be done by clicking on the trend appending buttons. In FIG. 5, the trend appending buttons 226 are located adjacent to the horizontal scroll bar 224 and are labeled with the words “previous” and “next.” In order to scroll further back in time, the clinician clicks on the trend appending button 226 marked “previous” and an additional quantity of data from the next previous time period is downloaded. In some embodiments, the additional quantity of data includes data for the next previous time period for an amount of time equal to two times the period of time which is visible on the screen in the view. Similarly, in order to scroll further forward in time, the clinician clicks on the trend appending button 226 marked “next,” signaling the data center to send the next most recent time period of data to the web page. In the example shown in FIG. 5, the trend appending button 226 marked “next” is grey, indicating that there is no more recent data available to be appended. In addition, the web page may include a skip-to-end button 228 to allow the clinician to skip ahead to the most recent data without scrolling through intervening downloads. In the example shown in FIG. 5, the skip-to-end button 228 is labeled with a double arrow head pointing to the right (similar to a fast-forward symbol on a media player) and is located near the bottom of the page, adjacent to the right end of the horizontal scroll bar 224. By using the skip-to-end button 228, the clinician can return to the most recent data view more quickly without scrolling through the interceding time frames and downloading data.

The clinician may adjust the window of time displayed on the web page by clicking on time window buttons 230. In the example shown in FIGS. 4-5, there are four time window buttons 230 located near the top of the page. The buttons provide four different time windows views including one day (labeled with the number “1”), one week (labeled with the number “7”), one month (labeled with the number “31”) and one year (labeled with the number “365”). By changing time views, the clinician is thus able to zoom in and out of areas of interest. In FIGS. 4 and 5, the time down button 230 marked with “7” is darkened to distinguish it from the other buttons, indicating that a one week view is being shown.

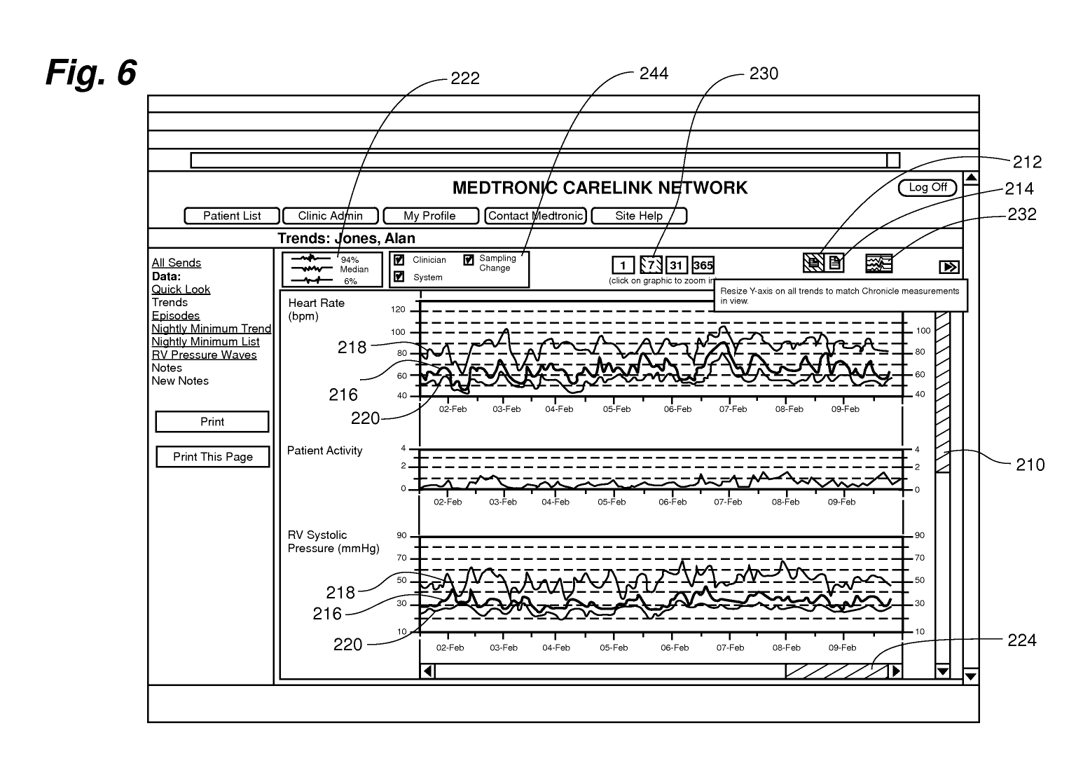

As the clinician scrolls horizontally over time with new data being appended, the data is presented in the same y-axis scale so that the data can be meaningfully compared to the data previously viewed. However, in some situations, the range in variation of values may change over time such that a particular y-axis scale may no longer be appropriate. As a result, the data may begin to appear too flat or may extend outside of the range of the y-axis. In order to change the y-axis to a more appropriate scale for the data being viewed, the clinician may click on a rescale button 232 shown in FIG. 6. Using scaling software including a scaling rules engine, the data center determines the appropriate scale for viewing the data on the screen and adjusts the y-axis appropriately. In the example shown in FIG. 6, the rescale button 232 is located to the right of the secondary trend button 214 of the webpage and is labeled with a mini version of trends.

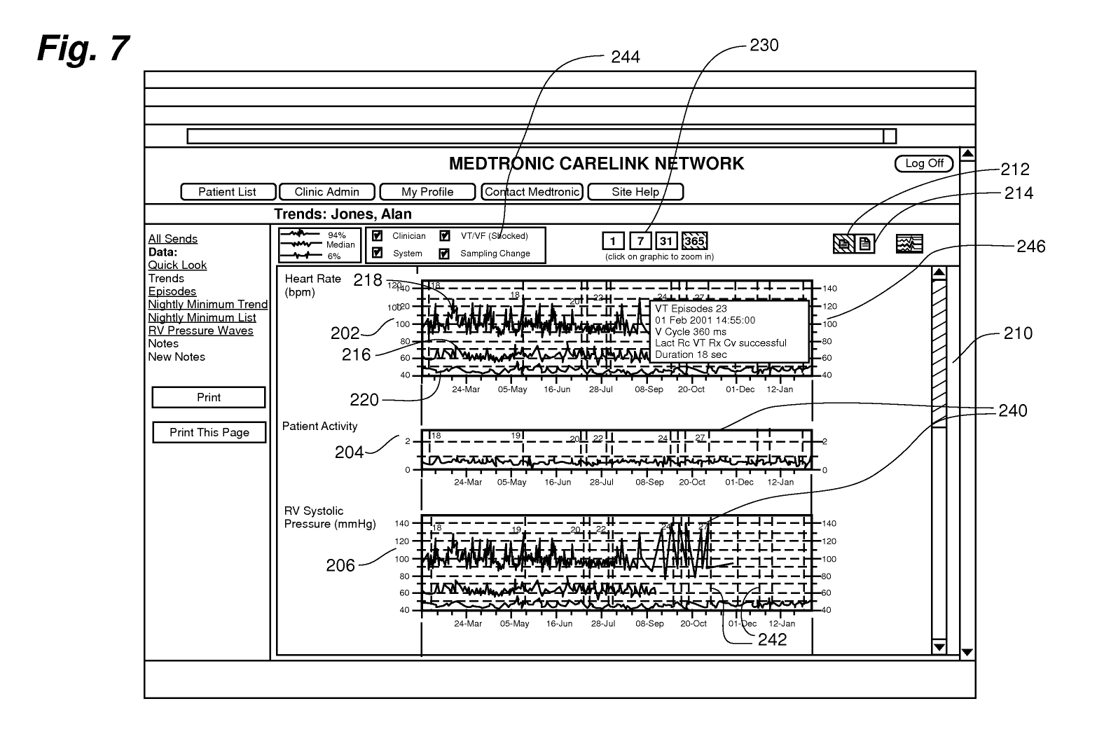

The data trends may optionally include annotations. For example, the clinician may add notes commenting about the data. Alternatively, the system may add notes commenting about the data or indicating the detection of an event such as a shock. So that the notes do not obscure the data, they are not typically visible on the data trends but rather the presence of a note is indicated by a note marker 240, shown in FIGS. 5 and 7, which may be a number and/or a symbol. When different types of notes are available, the different note types could be indicated on the data trend by the use of different symbols as note markers to represent different note types.

In the example shown in FIG. 7, the presence of a note is indicated by a horizontal line 242 passing through the data which is labeled with a note marker 240 which is both a number and a symbol. A note marker legend 244 is located in the upper left portion of the webpage correlating each note marker symbol to the type of note being represented. The types of notes may include, for example, clinician notes, system notes, ventricular tachycardia and ventricular fibrillation and shock delivery, and sampling change, indicating a change in the data sampling rate. Each type of note is represented by a different symbol and the symbol appears with the note number on the data trend.

The note markers may optionally be visible on the data trends or may be hidden, according to the clinician's preference. In FIG. 7, the note marker legend 244 includes a small checkbox adjacent to each type of note. These checkboxes may be clicked by the clinician to check and uncheck the note type. When the note type is checked, the presence of this note type is selected and it is visible on the data trend. However, when the note type is unchecked, the notes of that type are not selected and are hidden on the data trend. Other ways of selecting and un-selecting notes may also be used. In this way, the physician can choose to have the presence of all notes, no notes, or only selected types of notes visible on the data trend.

The clinician may view the note in a variety of ways. When the cursor hovers over the note marker 240, the note may appear as a note window 246 on top of the trend data, as shown in FIG. 7. Alternatively or additionally, the notes may be listed elsewhere on the webpage, such as at the bottom of the webpage, similar to a footnote, and may be identified by number corresponding to the note marker number.

Examples of notes which may be added to the trend data include text comments, such as by a clinician or by the system. The notes may also include other types of data besides text, such as images which relate to the trend data. The images may be cardiac images, for example, EMR images or echocardiographic images. The notes may also include data such as source material. The notes may also include links which can be opened to display related or relevant data, such as other trend displays or views. For example, a note may indicate the occurrence of an episode of ventricular tachycardia or ventricular fibrillation and may also include a link. The link may be clicked to open a view of the implantable cardiac device episode record including information about the delivery of shocks, the ECG recording, and/or other data.

In addition or alternatively to using notes, the trend viewer may identify clinically significant events by highlighting, shading, or otherwise marking a range of time on the trend lines. For example, the trend viewer may be programmed with threshold values for the data being displayed. These threshold values could be predetermined and/or they could be set by the clinician. When the value of the data exceeds a maximum threshold or falls below a minimum threshold, the data would be marked on the trend viewer to draw the clinician's attention to this portion of the data trend. Alternatively or additionally, the trend viewer may mark the detection and occurrence and duration of relevant events, such as atrial or ventricular fibrillation.

In addition to using thresholds for marking the data in the data viewer, such thresholds may also be used as warning levels. When the data exceeds a threshold, a warning may be sent to a clinician and/or to the patient. Such a warning system would be useful in implantable medical devices as well as hand held devices such as glucose monitors.

To assist the user, the data display may include tool tips. When the user rolls the mouse over a button, a pop-up tool tip message 250 may appear indicating what the function of the button is. An example of a pop-up tool tip message 250 is shown in FIG. 4, where a tool tip message is shown indicating the function of the primary and secondary trend button 214.

To assist the user to better assess and determine trends, the trend viewer may automatically generate a trend or ‘line of best fit’ for existing plotted data, and/or may be used to extrapolate the plot into the future for a period of time, such as a period of time specified by user input. In some embodiments, the system can calculate slope (rise/run) for a specified series or point. For example, the user may access the slope function, such as by highlighting the ‘slope function.’ The user may then click on two specific data points on the display, and the system will then calculate the corresponding slope (rate of change) between the two points. In some embodiments, the user may select date points, such as the rate of change according to a time period, and conduct sensitivity analyses.

What is claimed is:

1. A system for displaying trended data retrieved from a medical device, comprising:
a medical device which monitors and stores trended data over a first period of time for a patient associated with the medical device;
a data center; and
a relay device which receives and uploads trended data from the medical device to the data center, the data center merging the uploaded data with trended data received during previous data uploads to form a continuum of trended data over a second period of time longer than the first period.

2. The system of claim 1, wherein the trended patient data in the data center is accessible to clinicians as an internet webpage.

3. The system of claim 2, wherein the webpage is accessible to the clinician using a work station remote from the data center.

4. The system of claim 2, wherein the internet webpage allows a clinician to seamlessly view the trended data over time.

5. The system of claim 1, wherein the trended patient data includes hemodynamic data.

6. The system of claim 1, wherein the medical device is an implanted medical device which provides monitoring and therapy for a heart.

7. A system for displaying trended data retrieved from a medical device, comprising:
an implantable medical device which monitors and stores trended patient data;
a data center; and
a communications device located proximate to the implanted medical device which receives and uploads trended patient data from the medical device to the data center, the data center merging the uploaded data with trended patient data obtained during previous data uploads, the data center providing the merged data to clinicians as trended data on a web page.

8. The system of claim 7, wherein the trended data is displayed on a web page in a default view, and wherein the specific trended data displayed and the duration of time of the trended data displayed in the default view can be customized by a clinician.

9. The system of claim 7, wherein the data center downloads trended data for time periods greater than the time period visible on the webpage and wherein all of the downloaded trended data may be viewed by scrolling the webpage forward or backward.

10. The system of claim 9, wherein the data center downloads trended data for time periods equal to twice the time period visible on the webpage.

11. The system of claim 9, wherein a clinician may request that trended data be downloaded for an additional period of time prior to or following a previously downloaded time period visible on the webpage.

12. The system of claim 7, wherein the trended data on the web page includes roll over notes.

13. The system of claim 12, wherein the roll over notes include the numerical value of the data point or comments about the data.

14. The system of claim 13, wherein the comments are provided by the system or by a clinician.

15. The system of claim 12, wherein the presence of a roll over note is indicated on the webpage by a note marker and wherein the note marker indicates the type of roll over note.

16. The system of claim 15, wherein the webpage allows a clinician to select or unselect roll over notes to make the notes visible or hidden.

17. They system of claim 7, wherein the data center includes a scaling rules engine.

18. The system of claim 17, wherein the webpage allows a clinician to activate the scaling rules engine to resize the y-axis of the trended data.

19. A method for displaying trended data retrieved from a medical device comprising:
monitoring and storing trended data using an implanted medical device;
transmitting the trended data from the implanted medical device to an intermediate device;
uploading the trended data from the intermediate device to a data center;
merging the uploaded data with patient data obtained during previous data uploads; and
displaying the trended data on a web page upon demand by a clinician.

20. The method of claim 19, wherein the implanted medical device provides monitoring of a heart and wherein the trended data includes impedance values.
